(19) United States
(12) Patent Application Publication
(10) Pub. No.: US 2020/0009535 A1
(43) Pub. Date: Jan. 9, 2020

(54) SILVER CATALYST SYSTEM HAVING A REDUCED PRESSURE DROP FOR THE OXIDATIVE DEHYDROGENATION OF ALCOHOLS

(71) Applicant: BASF SE, Ludwigshafen am Rhein (DE)

(72) Inventors: Ekaterina TROUSSARD, Kaisten (CH); Roman Daniel PILZ, Ludwigshafen am Rhein (DE); Emil ROETH, Ludwigshafen am Rhein (DE); Marco BOSCH, Ludwigshafen am Rhein (DE); Susanne WEBER, Ludwigshafen am Rhein (DE); Peter LOECHER, Ludwigshafen am Rhein (DE); Daniel PFEIFFER, Ludwigshafen am Rhein (DE); Thomas HOLTMANN, Ludwigshafen am Rhein (DE)

(21) Appl. No.: 16/486,914

(22) PCT Filed: Feb. 14, 2018
(86) PCT No.: PCT/EP2018/053604

§ 371 Date: Aug. 19, 2019

(30) Foreign Application Priority Data
Feb. 24, 2017 (EP) 17157921.2

(51) Int. Cl. B01J 23/50; B01J 35/00; B01J 35/02; B01J 35/06; B01J 37/14; B01J 37/08; C07C 45/00

(52) U.S. Cl. CPC B01J 23/50; B01J 35/0006; B01J 35/0026; B01J 35/023; C07C 45/002; B01J 35/026; B01J 37/14; B01J 37/08; B01J 35/06

(57) ABSTRACT

The invention relates to a silver-comprising catalyst system for the preparation of aldehydes and/or ketones by oxidative dehydrogenation of alcohols, in particular the oxidative dehydrogenation of methanol to form formaldehyde, comprising a first catalyst layer and a second catalyst layer, wherein the first catalyst layer consists of a silver-comprising material in the form of balls of wire, gauzes or knitteds having a weight per unit area of from 0.3 to 10 kg/m² and a wire diameter of from 30 to 200 μm and the second catalyst layer consists of a silver-comprising material in the form of granular material having an average particle size of from 0.5 to 5 mm and the two catalyst layers are in direct contact with one another. The invention further relates to a corresponding process for the preparation of aldehydes and/or ketones, in particular of formaldehyde, by oxidative dehydrogenation of corresponding alcohols over a silver-comprising catalyst system.

The present invention relates to an improved silver-comprising catalyst system for the preparation of aldehydes and/or ketones by oxidative dehydrogenation of alcohols, in particular the oxidative dehydrogenation of methanol to form formaldehyde, comprising a first catalyst layer and a second catalyst layer, wherein the first catalyst layer consists of a silver-comprising material in the form of balls of wire, gauzes or knitteds having a weight per unit area of from 0.3 to 10 kg/m² and a wire diameter of from 30 to 200 μm and the second catalyst layer consists of a silver-comprising material in the form of granular material having an average particle size of from 0.5 to 5 mm and the two catalyst layers are in direct contact with one another. The invention further relates to a corresponding process for preparing aldehydes and/or ketones, in particular formaldehyde, by oxidative dehydrogenation of corresponding alcohols over a silver-comprising catalyst system.

The term oxidative dehydrogenation refers to the conversion of alcohols into corresponding aldehydes and/or ketones in the presence of an oxygen-comprising gas mixture, preferably oxygen, with at least part of the hydrogen formed reacting with oxygen to produce water. The reaction can take place either in the gas phase or in the liquid phase, with preference being given to carrying out the reaction in the gas phase.

The process for preparing formaldehyde by oxidation/dehydrogenation of methanol over a silver catalyst has been known for a long time (Ullmann's Encyclopedia of Industrial Chemistry, Weinhelm, 2016, Chapter “Formaldehyde”, pp. 1-34; Applied Catalysis A: General, Vol. 238 (2003), pp. 211-222, “Formaldehyde synthesis from methanol over silver catalysts”; Chemie Ingenieur Technik, Vol. 41 (1969), pp. 962-966, “Herstellung von Formaldehyd aus Methanol in der BASF”; Thesis “Mechanism and Modelling of the Partial Oxidation of Methanol over Silver”, A. Schlunke, University of Sydney, 2007, pp. 14-17).

Electrolytically produced silver crystals are usually used as catalyst. For this purpose, silver is oxidized anodically to form silver ions in an electrolytic cell and is cathodically reduced again to silver. The coarsely crystalline silver formed at the cathode is suitable as catalyst for the synthesis of formaldehyde from methanol. Particularly good results can be achieved using the fixed-bed catalysts described in DE 2322757 A. Suitable silver crystals are obtained, in particular, by means of the electrolysis described in DE 1166171 B. An aqueous silver nitrate solution is preferably used as electrolyte. This silver nitrate solution generally has a pH of from 1 to 4 and comprises from 1 to 5% by weight of silver. The pH is preferably set using nitric acid. The electrodes customarily used in the electrolysis of silver serve as electrodes. Suitable anodes are sacks into which the silver to be oxidized has generally been introduced as granules or as powder. Possible cathodes are, in particular, silver sheets. The electrolysis is preferably carried out at current densities of from 80 to 500 A per m² of cathode area and electrolyte temperatures of from 10 to 30° C. In order to achieve these current densities, voltages of from 1 to 15 volt are required in most electrolysis cells. It is advisable for the silver crystals formed at the cathode to be removed continually from the cathode.

Silver crystals having a particle size of from 0.2 to 5 mm are generally obtained. A one-off electrolysis is usually sufficient to obtain usable silver crystals. In general, the silver crystals are arranged to give starting silver catalyst fixed beds which consist of from 1 to 9 layers of silver crystals (known as “short layers”) having a total layer thickness of from 1 to 10 cm.

There are two process variants of the process for preparing formaldehyde by partial oxidation/dehydrogenation of methanol over a silver catalyst. In the BASF process (also referred to as “complete conversion process”), the methanol conversion is 96% or more. The residual amount of unreacted methanol remains in the product mixture. The proportion of methanol in the product is correspondingly low. This means that no removal and recirculation of methanol has to be carried out after the reaction. In the alternative process, the methanol conversion is in the range from 77 to 87%. The low conversion of methanol makes removal and recirculation of unreacted methanol necessary. Typical yields of formaldehyde in the BASF process are in the range from 89.5 to 90.5%, while the yields in the alternative process are typically in the range from 91 to 92%.

The use of silver crystal catalysts for the oxidative dehydrogenation of methanol to form formaldehyde is known. EP 624565 A discloses the preparation of formaldehyde by oxidative dehydrogenation of methanol over a silver catalyst fixed bed by passing a gas mixture comprising methanol, oxygen and dinitrogen oxide through the catalyst. EP 880493 A describes a process for preparing formaldehyde by oxidative dehydrogenation of methanol, wherein a gas mixture comprising methanol, oxygen, dinitrogen oxide and water is passed at a temperature of from 150 to 800° C. through a phosphorus-doped silver catalyst fixed bed. As a result of densification of the catalyst bed by intercrystalline transformation of silver, an increasing pressure drop occurs over time under the reaction conditions, and this has an adverse effect on the activity (Chemie Ingenieur Technik Vol. 41 (1969), pp. 962-966, “Herstellung von Formaldehyd aus Methanol in der BASF”). Furthermore, it is reported that silver gauzes grow together under reaction conditions and a “dendritic” growth is observed for the silver, leading to the pressure drop increasing to such an extent that continuation of the production process is no longer feasible (Catalyst Manufacture, 2nd Edition” (1995), Marcel Dekker, Inc., Vol. 20, pp. 203, “Oxidation of Methanol to Formaldehyde”).

Analyses of the course of the reaction and also of silver crystal catalyst samples taken from the reactor have shown that the increase in the pressure drop is associated with the formation of a sponge-like silver structure.

The formation of the sponge-like structure (also referred to as dendritic growth of silver) is caused by the reaction conditions and brings about a significant increase in the surface area of silver which is available for the reaction. The formation of the sponge-like structure does not stop during the production process but instead leads to ever finer silver filaments and ever narrower free spaces in the sponge structure. The transformation of at least part of the silver crystals of these catalysts, which typically consist of electrolytic silver crystals having sizes in the range from 0.2 mm to 5 mm, into the sponge-like structure explains the observation that the conversion and selectivity initially increase during operation in the case of a freshly installed silver crystal catalyst. On the other hand, the ever finer sponge structure leads to an increase in the pressure drop over the catalyst, as a result of which the proportion of unselective gas-phase reactions increases and the selectivity to the product (for example formaldehyde) decreases. Accordingly, an increase in the input number (defined as metric tons of methanol required to produce one metric ton of formaldehyde (based on a formaldehyde concentration of 100%)) occurs over the course of the catalyst operating life in processes for preparing formaldehyde in which silver crystal catalysts are used. A similar situation is observed when silver catalysts are used in other forms known in the literature, for example in the form of fibers, gauzes, knitteds or foams. In addition to the decrease in selectivity and yield, a higher pressure drop also leads to higher costs for compression of the reaction gas, In addition, the reactor has to be run down and the catalyst system replaced above a certain pressure drop when the technical limitation of the plant (e.g. maximum compressor power) has been reached. More frequent replacement of the catalyst system reduces the production capacity of the plant and incurs costs for the removal and installation of the catalyst system and also the recovery of the silver. The typical operating life of silver crystal catalysts is from 0.5 to 4 months.

U.S. Pat. No. 4,076,754 describes a two-stage process for preparing formaldehyde from methanol, air and water. A catalyst in which 40 layers of superposed silver gauzes made of silver wire having a diameter of 0.014 inch (corresponding to 350 μm) and having a mesh opening of 1.25 mm (“20 mesh silver gauze”) have been applied to a layer of silver crystals is used here.

DE 2829035 A describes a catalyst made of catalytically active metal fibers which consist of silver, platinum, rhodium, palladium or an alloy based on one of these metals, with the metal fibers being joined to one another in a felt-like manner as in a needle composite material. The catalyst can be used for the oxidation of ammonia or the preparation of hydrocyanic acid or formaldehyde. The cross section of a band-shaped fiber can be rectangular with the dimensions 50 and 100 μm, and the length can be in the range from 10 cm to 1 m.

DE 3047193 A describes a catalyst composed of silver or a silver alloy. The catalyst body is produced by a melt spinning process or melt extraction process. For example, wavy catalyst bodies which are about 1 cm long and are thus rather short-fibered are obtained from a strip having a width of from 1 to 2 mm and a thickness of from 50 to 60 μm by crimping (embossing of a wave profile) and cutting.

EP 0218124 B describes a process for producing a supported catalyst in the form of a support material provided with a silver layer as active component, in particular a gauze-, woven fabric- or foil-like support material, wherein the silver layer is polycrystalline and has been formed by vapor deposition of metallic silver on the support material and subsequent thermal treatment at from 200 to 800° C.

WO 2012/146528 describes a process for preparing C₁-C₁₀-aldehydes by oxidative dehydrogenation of C₁-C₁₀-alcohols over a shaped catalyst body which is obtainable by three-dimensional deforming and/or arrangement in space of silver-comprising fibers and/or threads, wherein the average diameter or the average diagonal length of an essentially rectangle or square cross section of these silver-comprising fibers and/or threads is in the range from 30 to 200 μm. The description refers to the use of the combination of silver-comprising fibers and/or threads and Ag crystal particles as catalyst. However, there is no teaching as to how the two materials are combined in order to bring about the advantageous effects of the present invention.

WO 2015/086703 describes a process for producing metal foam bodies, in which the metal M(x) is present either as pure substance or as a mixture with metal M(y) and the metals M(x) and M(y) are selected from a group consisting of nickel, chromium, cobalt, copper and silver, and also the use of such metal foam bodies as catalyst for the preparation of formaldehyde from methanol. A combination of silver-comprising metal foams with other silver-comprising shaped catalyst bodies for the preparation of formaldehyde from methanol is not mentioned in this document.

U.S. Pat. No. 3,959,383 describes a two-stage process in which a silver catalyst consisting of fibrous electrolytic silver, crystal-like electrolytic silver or silver on a support such as aluminum oxide is used in the first stage and a crystal-like electrolytic silver catalyst is used in the second stage. In the two-stage process, additionally, the first and second stages are in each case carried out in physically separate reactors under adiabatic conditions at from 575° C. to 650° C. and from 600° C. to 700° C., respectively, and the temperature of the reaction gas after the first stage is actively cooled to <300° C. by means of a heat exchanger before being fed together with additional oxygen into the second stage.

CN 100435943 C describes a catalyst system for the conversion of methanol into formaldehyde, which is made up of two parallel layers, with the first layer consisting of fibrous electrolytic silver and the second layer consisting of crystal-like electrolytic silver. The proportions are from 10 to 35% by weight for the first layer and from 65 to 90% by weight for the second layer. The particle size of the crystal-like electrolytic silver is in the range from 8 to 24 mesh (corresponding to from 2.38 to 0.74 mm) and that of the fibrous electrolytic silver is in the range from 32 to 40 mesh (corresponding to from 0.55 to 0.40 mm).

It is an object of the present invention to provide an improved silver-comprising catalyst system for the preparation of C₁-C₁₀-aldehydes and/or -ketones by oxidative dehydrogenation of the corresponding C₁-C₁₀-alcohols, in particular the oxidative dehydrogenation of methanol to formaldehyde, and a corresponding production process in which the pressure drop and in particular the increase in the pressure drop caused by the catalyst system is low under the reaction conditions and the selectivity is high, and at the same time a high conversion is achieved. Due to these improved properties of the catalyst system, the time on stream of the plant is increased, the proportion of unselective secondary reactions in the gas phase is decreased and the costs for compression of the fresh gas mixture is reduced. Compared to conventional silver crystal catalysts, the pressure drop and the pressure drop increase under the reaction conditions over the entire catalyst system should be at a lower level at at least the same activity and/or selectivity.

The object is achieved by provision of the silver-comprising catalyst system of the invention and of the process of the invention for the preparation of aldehydes and/or ketones by oxidative dehydrogenation of corresponding alcohols over such a silver-comprising catalyst system.

The silver-comprising catalyst system of the invention comprises
a first catalyst layer A composed of a silver-comprising material which is present in a form consisting of wires and selected from the group consisting of balls of wire, gauzes and knitteds and
   a second catalyst layer B composed of a silver-comprising material which is present in the form of granular material having an average particle size in the range from 0.5 to 5 mm,
   with the catalyst layer B being in direct contact with the catalyst layer A,

wherein the silver-comprising material of the catalyst layer A
has a weight per unit area in the range from 0.3 to 10 kg/m² for the entire catalyst layer A and
a wire diameter in the range from 30 to 200 μm.

The silver-comprising material of the catalyst layer A is present in a form selected from the group consisting of balls of wire, gauzes and knitteds, preferably in the form of gauzes or knitteds.

The silver-comprising material of the catalyst layer A is preferably electrolytic silver, preferably in a purity (silver content in % by weight) of ≥98%, preferably ≥99%, particularly preferably ≥99.9% and in particular ≥99.99%.

The silver-comprising material of the catalyst layer B is preferably electrolytic silver, preferably in a purity (silver content in % by weight) of ≥30%, preferably ≥50%, particularly preferably ≥90%, very particularly preferably ≥99.9% and in particular ≥99.99%.

In a particular embodiment, the silver-comprising material of the catalyst layer A and/or the catalyst layer B is phosphorus-doped.

The balls of wire, gauzes and knitteds of the invention consist of wires. These wires are silver-comprising fibers or threads, preferably silver-comprising fibers or threads having an essentially circular cross section, particularly preferably silver-comprising fibers or threads having a circular cross section. The silver-comprising fibers of the invention generally have a length in the range from 1 mm to 100 mm; the silver-containing threads of the invention can in theory have an infinite length, but in practice they generally have a length of from a few centimeters to a number of kilometers.

Silver-comprising fibers or threads are known to those skilled in the art, are commercially available and are used, for example, as electrical conductor material, in high-value textiles or in corrosion-resistant, sensor-type applications (e.g. pH determination). The three-dimensional deformation and/or arrangement of the silver-comprising fibers or threads in space can be carried out in a disordered or ordered manner.

This ordered deformation and/or arrangement of the silver-comprising fibers of the invention or preferably silver-comprising threads of the invention leads to the balls of wire according to the invention. They can, for example, be produced by the fibers or threads (optionally also arrangements of fibers or threads such as gauzes or knitteds) being packed to give a statistically irregularly arranged ball of wire and subsequently be compressed further under applied pressure to the desired ball of wire density or the desired proportion of voids in the ball of wire. In these balls of wire, the silver-comprising fibers or threads of the invention are arranged irregularly in space and can also be hooked together in a felt-like manner and in this way gain, for example, their particular mechanical stability.

The ordered deformation and/or arrangement of the silver-comprising fibers of the invention or preferably silver-comprising threads of the invention leads to essentially regular and ordered structures having periodically repeating unit cells, for example meshes or holes. Well-suited methods for ordered deformation and/or arrangement of the silver-comprising fibers or preferably silver-comprising threads in space are knitting, weaving or the like, optionally with subsequent densification. Inventive structures composed of silver-comprising fibers or preferably silver-comprising threads which can be obtained in this way are knitteds or gauzes, preferably having a mesh opening in the range from 300 to 50 mesh (corresponding to 80 to 500 μm), preferably in the range from 300 to 100 mesh (corresponding to from 80 to 250 μm). The mesh opening of gauzes and knitteds can be determined in accordance with the standard DIN ISO 9044 (August 1999, “Industrial woven wire cloth”). The term “gauze” as used for the purposes of the present invention encompasses all woven forms customary in industry, for example fine gauzes.

The silver-comprising knitteds, gauzes or gauze arrangements according to the invention or the silver-comprising balls of wire according to the invention generally have a density in the range from 2 to 4 g/cm³, preferably in the range from 3 to 4 g/cm³. The density generally corresponds to a proportion of voids in the silver-comprising knitteds, gauzes, gauze arrangements or balls of wire according to the invention in the range of preferably from 60 to 80%, in particular in the range from 60 to 75%. Such a proportion of voids is also advantageous because “lighting-off” of the catalytic oxidation/dehydrogenation of the alcohol at preferably low temperatures, for example 350° C. and less, advantageously the range from 200 to 350° C., is ensured. The silver-comprising knitteds, gauzes, gauze arrangements or balls of wire according to the invention are usually preheated until the reaction of oxidative dehydrogenation, (for example of methanol to formaldehyde) starts. The reaction is then generally self-sustaining under adiabatic conditions. The abovementioned density and the proportion of voids of/in the silver-comprising knitteds, gauzes, gauze arrangements or balls of wire of the invention is determined as follows: A body having a known geometry is weighed. The ratio of its mass to the volume taken up by it determines the density. The ratio to the mass of a body having the same geometric size and made of the same material defines the proportion of voids.

Foams are a further structure used for silver catalysts. A silver-comprising foam is a porous metallic structure. It preferably has a pore diameter in the range from 100 μm to 5000 μm, in particular from 200 μm to 1000 μm, an apparent density in the range from 300 kg/m³ to 1200 kg/m³, a specific geometric surface area in the range from 100 m²/m³ to 20 000 m²/m³ and a porosity in the range from 0.50 to 0.95. The pore diameter is determined by means of Visiocell analysis (Recticel, “The Guide 2000 of Technical Foams”, Book 4, Part 4, pp. 33-41). In particular, the pore diameter is determined by optical comparison of the cell diameter of a selected cell of the metal foam body with the aid of calibrated ring sizes, imaged on a sheet of transparent paper which is laid on the metal foam body. This measurement is carried out for at least 100 different cells, with the average of these measurements giving the pore diameter. The apparent density is determined in accordance with DIN EN-ISO 845 (October 2009, “Cellular plastics and rubbers—Determination of apparent density”). The specific geometric surface area (GSA) and the porosity are determined by the method described in WO 2015/086703. Metal foams are usually produced as follows: (1) provision of an organic porous polymer foam, (2) deposition of at least one metal or metal alloy M(x) on the porous organic polymer foam, (3) combustion of the porous organic polymer foam in order to obtain the metal foam body and (4) deposition by means of electroplating of the metal layer composed of a metal or metal alloy M(y) on at least part of the surface of the metal foam body. The deposition of metal or metal alloy M(x) on the porous organic polymer foam can be carried out in various ways, for example by electroplating, CVD, metal-organic CVD (MOCVD) or by means of a slurry process. For electroplating, the porous polymer is made electrically conductive so that it is suitable for the electrochemical process. The deposition of a first metal layer comprising a metal or a metal alloy M(x1) is preferably carried out by means of a chemical or physical vapor deposition process, for example by a sputtering process, and the application of a second metallic layer comprising a metal or a metal alloy M(x2) is carried out by electroplating, where M(x1) and M(x2) are able to be identical or different. The first metallic layer generally serves to make the surface of the porous organic polymer electrically conductive. Accordingly, the first metal layer can be quite thin as long as it has a sufficient electrical conductivity. In general, it is sufficient for this first metal layer to have a thickness in the order of a few atoms. Preference is given to the first metal layer M(x1) having an average thickness of up to 0.1 μm and the second metal layer M(x2) having an average thickness in the range from 5 to 50 μm. Numerous porous organic polymers can be used in the production of metal foam bodies. Preference is given to using organic polymers having open pores. In general, the porous organic polymer foam is selected from the group consisting of polyurethane foam, polyethylene foam and polypropylene foam. Preference is given to using a porous polyurethane foam, which leads to a particularly advantageous open-celled metal foam body. The production of silver-comprising foams is described, for example, in WO 2007/121659, CN 104577135 A, KR 100921399 B and WO 2015/086703. WO 2007/121659 describes a process for producing metal foam body plates having a three-dimensional metal structure, where the layer thickness of the plates is in the range from 0.3 to 10 mm, the density is in the range from 100 to 5000 g/m², the porosity is less than 99.5% and the metal can be selected from the group consisting of silver and copper. CN 104577135 A describes a process for producing silver foams by coating a polymer sponge with silver by means of a sputtering process, followed by electrochemical coating of the conductive polymer sponge with silver and a thermal after-treatment in which the polymer material is decomposed. After reduction, a three-dimensional silver foam is obtained using this method. KR 100921399 B describes a process for producing open-celled silver foams which do not comprise any platinum contamination, by pretreatment of a polyurethane foam with a sodium hydroxide solution, followed by electrolytic application of silver and heat treatment of the silver-comprising polyurethane foam at temperatures in the range from 800 to 950° C. The production of the silver-comprising foam of the invention is preferably carried out as described in WO 2015/086703. The silver-comprising foam can also be treated as described in DE 4424157 A in order to optimize the anisotropic properties, in particular in respect of the thermal and electrical conductivity, for use as catalyst for preparing formaldehyde from methanol.

The granular material according to the invention is particulate material consisting of small, generally irregularly shaped, solid particles, preferably electrolytically produced silver crystals.

The wire diameter in the case of balls of wire, gauzes and knitteds corresponds to the average diameter or the average diagonal length of the wires (fibers or threads) of which the balls of wire, gauzes or knitteds are made. The average diameter (for an essentially round cross section) of the silver-comprising fibers or threads is determined by means of a micrometer in accordance with DIN ISO 4782 (October 1993, “Metal wire for industrial wire screens and woven wire cloth”). The average diagonal length (for an essentially rectangular or square cross section) of the silver-comprising fibers or threads is likewise determined in an analogous way by means of a micrometer (measurement of height and width and calculation of the diagonal length).

The average particle size of the granular material can be determined by the method set forth in DIN 66165-1 and 66165-2 (August 2016) (“Particle size analysis—sieve analysis”).

When silver-comprising materials in various forms (for example a plurality of different gauzes or different granular materials) are used for the catalyst layer A or B, then each of the silver-comprising materials used has to conform to the wire diameter according to the invention or the average particle size according to the invention.

The wire diameter of the silver-comprising material of the catalyst layer A is in the range from 30 μm to 200 μm, preferably in the range from 30 μm to 150 μm and in particular in the range from 30 μm to 70 μm. The threads or fibers of these balls of wire, gauzes or knitteds preferably have an essentially circular cross section and the threads or fibers of these balls of wire, gauzes or knitteds particularly preferably have a circular cross section.

The average particle size of the silver-comprising material of the catalyst layer B is in the range from 0.5 mm to 5 mm, preferably in the range from 0.75 mm to 4 mm and in particular in the range from 1 mm to 3 mm.

The weight per unit area of the silver-comprising material of the catalyst layer A is the mass of this material for the total catalyst layer A divided by the area of the layer and averaged over the entire catalyst bed. It is in the range from 0.3 to 10 kg/m², preferably in the range from 0.3 to 4 kg/m², particularly preferably in the range from 0.3 to 3 kg/m², very particularly preferably in the range from 0.6 to 3 kg/m² and in particular in the range from 1 to 3 kg/m². The weight per unit area at each individual point in the catalyst bed preferably varies by not more than +/−30%, based on the average weight per unit area.

From a practical point of view, it is advantageous for the layer height of the catalyst layer B to be at least 10 mm, preferably at least 15 mm, particularly preferably at least 20 mm and in particular at least 25 mm, in order to obtain a very uniform height distribution of the catalyst layer B over the cross section of the catalyst bed diameter. In a particular embodiment, the layer height of the catalyst layer B varies, within the preferred heights indicated, over the catalyst bed diameter with the objective of obtaining an optimally uniform flow through the entire catalyst bed, so that the catalyst bed is optimally utilized.

The catalyst layers A and B can in each case consist of various sublayers which comprise different silver-comprising materials or have different shapes of the silver-comprising materials (for example sublayers of catalyst layers B comprising granular materials of different particle sizes or, for example in the case of gauzes, sublayers of catalyst layers A comprising gauzes having different mesh openings). In a particular embodiment, the catalyst layers A and B are in each case not made up of sublayers.

In the silver-comprising catalyst system of the invention, the major part, preferably at least 80% by weight, of the catalyst layer A is preferably converted under the reaction conditions into a finely structured silver layer on the silver-comprising material of the catalyst layer B within 30 days, particularly preferably within 15 days and in particular within 5 days. As an alternative, this conversion can also take place under separate activation conditions with the objective of shortening the time, preferably to two days or less. The silver-comprising material of the catalyst layer B has such a configuration that, after deposition of the finely structured silver layer, sufficient free space is present for the pressure drop over the silver-comprising catalyst system not to increase significantly. This means, in particular, that the increase in the pressure drop of the catalyst system after activation is less than 30%. The material of the catalyst layer A serves as sacrificial material for the formation of the finely structured silver layer on the silver-comprising material of the catalyst layer B. It is therefore necessary in the silver-comprising catalyst system of the invention for the catalyst layer A to be in direct contact with the catalyst layer B (catalyst layers A and B touch one another). The activated, silver-comprising catalyst system obtainable in this way, in which the catalyst layer A has been mostly converted into a finely structured silver layer on the silver-comprising material of the catalyst layer B, enables good activity and selectivity for the preparation of aldehydes and/or ketones by oxidative dehydrogenation of corresponding alcohols while at the same time the pressure drop and the pressure drop increase under the reaction conditions over this catalyst system is particularly low. For this purpose, it is important for a suitable form of material (e.g. gauze) to be selected for the catalyst layer A and for the wire diameter and the weight per unit area to be in the ranges according to the invention. If the wire diameter or the weight per unit area of the catalyst layer A is too high, or if an unsuitable form is used, a significant proportion is not converted into the finely structured silver layer. This remaining proportion sinters and thus leads to an increased pressure drop or pressure drop increase. If the wire diameter or the weight per unit area of the catalyst layer A is too low, or an unsuitable form is used, a sufficiently thick finely structured silver layer is not formed and the catalyst activity is too low. If the average particle size in the catalyst layer B is too low, or an unsuitable form is used, the finely structured silver layer being formed from the silver-comprising material of the catalyst layer A clogs the free spaces between the silver-comprising material in the catalyst layer B excessively and an increased pressure drop or pressure drop increase occurs. If the average particle size of the catalyst layer B is too large, or an unsuitable form is used, the formation of the finely structured silver layer is insufficient and the catalyst activity is too low.

For the purposes of the present invention, reaction conditions are the passing of the feed stream comprising one or more C₁-C₁₀-alcohols and one or more oxidizing agents through the catalyst system at a temperature in the range from 350 to 750° C., a space velocity in the range from 36 000 h⁻¹ to 1 800 000 h⁻¹ and an inflow velocity in the range from 0.1 m s⁻¹ to 15 m s⁻¹. The one or more oxidizing agents are preferably used in a proportion of from 0.01 to 9% by weight based on the total feed stream. In a particular variant, the feed stream comprises inert gases such as nitrogen and/or additives such as halogenated hydrocarbons, for example C₂H₄Cl₂ or C₂H₂Cl₂, where the additives are preferably used in the ppm range (for example not more than 500 ppm by weight) based on the feed stream.

For the purposes of the present invention, activation conditions are the passing of a mixture comprising oxygen or one or more other oxidizing agents and an oxidizable material (proton donor) such as alcohol or hydrogen through the catalyst system at a temperature in the range from 350° C. to 750° C., a space velocity in the range from 36 000 h⁻¹ to 1 800 000 h⁻¹ and an inflow velocity in the range from 0.1 m s⁻¹ to 15 m s⁻¹. The one or more oxidizing agents are preferably used in a proportion of from 0.01 to 9% by weight based on the total feed stream. In a particular variant, hydrogen is used as oxidizable material and air is used as oxidative material, preferably in a volume ratio of not more than 1:25. In a particular variant, the feed stream comprises inert gases such as nitrogen and/or additives such as halogenated hydrocarbons, for example C₂H₄Cl₂ or C₂H₂Cl₂, where the additives are preferably used in the ppm range (for example not more than 500 ppm by weight) based on the feed stream.

The invention also provides activated catalyst systems which are formed by reaction of the catalyst system of the invention under reaction conditions or activation conditions, preferably under reaction conditions or activation conditions prevailing for at least two days, particularly preferably at least five days, preferably in such a way that at least 50% by weight, particularly preferably at least 80% by weight, of the silver-comprising material of the catalyst layer A is converted into a finely structured silver layer of the silver-comprising material of the catalyst layer B.

The invention also provides a process for the preparation of C₁-C₁₀-aldehydes and/or -ketones by oxidative dehydrogenation of the corresponding C₁-C₁₀-alcohols, wherein a feed stream comprising one or more C₁-C₁₀-alcohols and one or more oxidizing agents is passed at temperatures in the range from 350 to 750° C., preferably in the range from 400 to 750° C., in particular in the range from 450 to 750° C., very particularly preferably in the range from 550 to 750° C., in particular in the range from 595 to 710° C., through one or more catalyst systems according to the invention in such a way that the feed stream in each case flows firstly through the catalyst layer A (if still present after activation) and immediately afterward through the catalyst layer B of the one or more catalyst systems. The invention also provides, in particular, a process for the preparation of C₁-C₁₀-aldehydes and/or -ketones by oxidative dehydrogenation of the corresponding C₁-C₁₀-alcohols, wherein a feed stream comprising one or more C₁-C₁₀-alcohols and one or more oxidizing agents is passed at temperatures in the range from 350 to 750° C., preferably in the range from 400 to 750° C., particularly preferably in the range from 450 to 750° C., very particularly preferably in the range from 550 to 750° C., in particular in the range from 595 to 710° C., through a catalyst system according to the invention in such a way that the feed stream flows firstly through the catalyst A (if still present after activation) and immediately afterward through the catalyst layer B of the catalyst system. The catalyst system of the invention is the activated catalyst system in which the silver-comprising material of the catalyst layer A has been converted in its entirety or in part into a finely structured silver layer on the silver-comprising material of the catalyst layer B, or the original catalyst system before such a conversion of the catalyst layer A.

The invention preferably provides a particular embodiment of the process of the invention in which formaldehyde is the C₁-C₁₀-aldehyde formed, methanol is the C₁-C₁₀-alcohol used and the feed stream is passed at temperatures in the range from 550 to 750° C., in particular in the range from 595 to 710° C., through the one or more catalyst systems of the invention. Such a process is particularly advantageous for the preparation of formaldehyde in complete conversion processes in which a high selectivity and at the same time a high conversion are necessary.

The process of the invention is preferably carried out in a reactor. The reactor is usually vertical and the feed stream is usually passed from the top downward through the reactor. Such reactors and process procedures are described, for example, in EP 467169 A, DE 2444586 A and EP 150436 A. The cross-sectional area of the reactor and of the catalyst system are usually selected so as to be the same.

In a particular embodiment of the process of the invention, the feed stream flows through a catalyst system according to the invention (reaction zone).

In a particular embodiment of the process of the invention, the feed stream flows through a plurality of catalyst systems according to the invention (reaction zones) which are “connected in series”. This connection in series can be realized in one reactor or in a reactor cascade.

The catalyst system in which the feed stream is reacted is usually used statically on a support device. Such support devices are known, for example gratings, baskets or perforated plates or strong meshes made of various materials, preferably metals, for example stainless steel or silver.

The space velocity for the process of the invention or for the reaction or activation conditions according to the invention is usually in the range from 36 000 h⁻¹ to 1 800 000 h⁻¹, preferably from 50 000 h⁻¹ to 1 000 000 h⁻¹, particularly preferably from 60 000 h⁻¹ to 500 000 h⁻¹ and in particular from 70 000 h⁻¹ to 300 000 h⁻¹. The space velocity (gas hourly space velocity=GHSV) having a unit h⁻¹, is defined as the ratio of the volume stream which flows through the catalyst bed (under standard conditions (p=1 bar, T=273.15 K)) to the catalyst volume in the reactor.

The average inflow velocity for the process of the invention or for the reaction or activation conditions according to the invention is usually in the range from 0.1 m s⁻¹ to 15 m s⁻¹, preferably from 0.2 m s⁻¹ to 5 m s⁻¹, particularly preferably from 0.3 m s⁻¹ to 3 m s⁻¹ and in particular from 0.5 m s⁻¹ to 2 m s⁻¹. The average inflow velocity having the unit m s⁻¹ is defined as the ratio of the volume stream which flows through the catalyst bed (under standard conditions (p=1 bar, T=273.15 K)) to the cross-sectional area of the catalyst system.

The pressure difference over the catalyst bed (catalyst system or, in the case of a plurality of catalyst systems connected in series, the totality of all catalyst systems), measured under the reaction conditions, in the process of the invention is usually in the range from 10 mbar to 700 mbar, preferably from 20 mbar to 500 mbar and particularly preferably from 30 mbar to 300 mbar. The increase in the pressure difference under reaction conditions over time at the same space velocity is usually in the range from 0.01 mbar per day to 10 mbar per day, preferably from 0.01 mbar per day to 3 mbar per day and particularly preferably from 0.01 mbar per day to 1 mbar per day, in the process of the invention.

The feed stream is preferably gaseous.

The process is preferably carried out continuously.

Possible C₁-C₁₀-alcohols for the process of the invention or for the reaction conditions according to the invention are alcohols having from 1 to 10 carbon atoms and one or more, preferably from 1 to 3, OH groups. The alcohols preferably have one or two OH groups, very particularly preferably one OH group. The alcohols have at least one secondary or primary OH group, preferably at least one primary OH group. The alcohols can be aliphatic, linear, branched or cyclic and can comprise one or more C-C double or triple bonds in the molecule. They can be aliphatic alcohols or aralkyl alcohols, with preference being given to aliphatic alcohols. Preference is given to primary alcohols or in the case of dihydric alcohols vicinal C₁-C₁₀-diols

The C₁-C₁₀-alcohols are preferably selected from the group consisting of methanol, ethanol, 1-propanol, isopropanol, n-butanol, isobutanol, sec-butanol, 1,2-ethanediol (ethylene glycol), 1,2-propanediol, 1,3-propanediol, 2-(2-hydroxyethoxy)ethanol (diethylene glycol), allyl alcohol, 3-methyl-2-butenol (prenol) and 3-methyl-3-butenol (isoprenol). A particularly preferred C₁-C₁₀-alcohol is methanol.

Possible C₁-C₁₀-aldehydes and -ketones are the aldehydes and ketones which can be obtained from the abovementioned C₁-C₁₀-alcohols by oxidative dehydrogenation. The aldehydes can have one or more aldehyde groups in the molecule and preferably have one or two aldehyde groups, in particular one aldehyde group, in the molecule. Examples of C₁-C₁₀-aldehydes according to the invention are formaldehyde (methanal), glyoxal (ethanedial), 2-hydroxyethanal (glycol aldehyde), 2-(2-hydroxyethoxy)ethanal, 3-methyl-2-butenal (prenal) and 3-methyl-3-butenal (isoprenal).

As oxidizing agent for the process of the invention or for the reaction or activation conditions according to the invention, it is possible to use either pure oxygen or preferably oxygen-comprising gas mixtures such as air or else other oxidizing gases such as nitrogen oxide (NO), nitrogen dioxide (NO₂), dinitrogen oxide (N₂O), dinitrogen tetraoxide (N₂O₄), or mixtures thereof.

In a particular embodiment of the process of the invention, methanol is converted into formaldehyde (methanal). Suitable starting materials for this purpose are pure methanol, technical-grade methanol, crude methanol produced by a high- or low-pressure process or advantageously mixtures thereof with water. The proportion by mass of methanol of such aqueous mixtures is advantageously from 30 to 99% by weight, preferably from 45 to 97% by weight, particularly preferably from 60 to 95% by weight, preferably from 70 to 90% by weight. In a particular embodiment, crude methanol which has been purified by the methods described in DE 1277834 B, DE 1235881 C or DE 136318 C by removal of a low-boiling fraction or by treatment with oxidizing agents and/or alkalis is used.

In processes for preparing formaldehyde, oxygen and methanol are advantageously used in a molar ratio of from 0.1:1 to 1.0:1, preferably from 0.25:1 to 0.6:1, particularly preferably from 0.35:1 to 0.5:1.

In the process of the invention for preparing formaldehyde, the methanol is preferably fed in vapor form, advantageously in a mixture with water vapor and optionally with an inert gas, into the reactor space. A suitable inert gas for the process is, for example, nitrogen, and the proportion of inert gas based on the gaseous methanol and water mixture can be varied and is usually in the range from 0.1 to 30% by volume, preferably from 0.15 to 20% by volume, particularly preferably from 0.2 to 10% by volume, preferably from 0.3 to 5% by volume.

In the process of the invention for preparing formaldehyde, the above-described reaction mixture (feed stream) is generally introduced at a temperature in the range from 50 to 200° C. and usually at an absolute pressure in the range from 0.5 bar to 2 bar, preferably from 1.0 to 1.7 bar, into the reactor.

It is advantageous in the process of the invention for the reaction gases leaving the reaction zone to be cooled, e.g. to temperatures in the range from 50 to 350° C., within a few seconds, for example within not more than 10 seconds. In the process of the invention for preparing formaldehyde, the cooled gas mixture is then advantageously fed into an absorption tower in which the formaldehyde is scrubbed out from the gas mixture by means of water or an aqueous formaldehyde-urea solution, advantageously in countercurrent. Specific variants of the generally known process for preparing formaldehyde, which can also be employed in the process of the invention, are described in DE 2444586 A, DE 2451990 A, EP 83427 A and EP 150436 A.

Compared to conventional catalyst systems or processes, the catalyst system of the invention and the process of the invention allow a smaller pressure drop and/or pressure drop increase at the same or better yield and selectivity in the oxidative dehydrogenation of C₁-C₁₀-alcohols to form the corresponding aldehydes or ketones, in particular the oxidative dehydrogenation of methanol to form formaldehyde. The catalyst system of the invention and the process of the invention thus make longer catalyst operating lives possible. At the same time, the catalyst system of the invention and the process of the invention make comparable or improved yields and selectivity possible in the oxidative dehydrogenation at a decreased mass of catalyst.

### FIGURES

FIG. 1: Schematic depiction of the arrangement of the catalyst system of the invention for the oxidative dehydrogenation of methanol to formaldehyde with a catalyst bed consisting of a first catalyst layer A (optionally in the form of a plurality of sublayers) and a second catalyst layer B optionally in the form of a plurality of sublayers) on a support C. Here, the feed stream (the fresh gas) X comprising methanol, oxygen, water and optionally nitrogen and formaldehyde flows through the catalyst bed and gives the reaction gas Y comprising methanol, water, hydrogen, carbon monoxide, carbon dioxide, formaldehyde and possibly nitrogen and oxygen.

FIG. 2: Schematic depiction of the experimental setup used for the examples. The starting materials air (1), nitrogen (2), deionized water (3) and methanol (4) are fed into a reactor column (5) having a preheating section (5a), catalyst packing (5b), electric heating (6) and quench cooler (7). The product mixture formed is fed into an absorption column (8) and from the bottom of the absorption column (9) is transferred into a condenser (10). Finally, the product is purified in a phase separator (11) with cryostat (12).

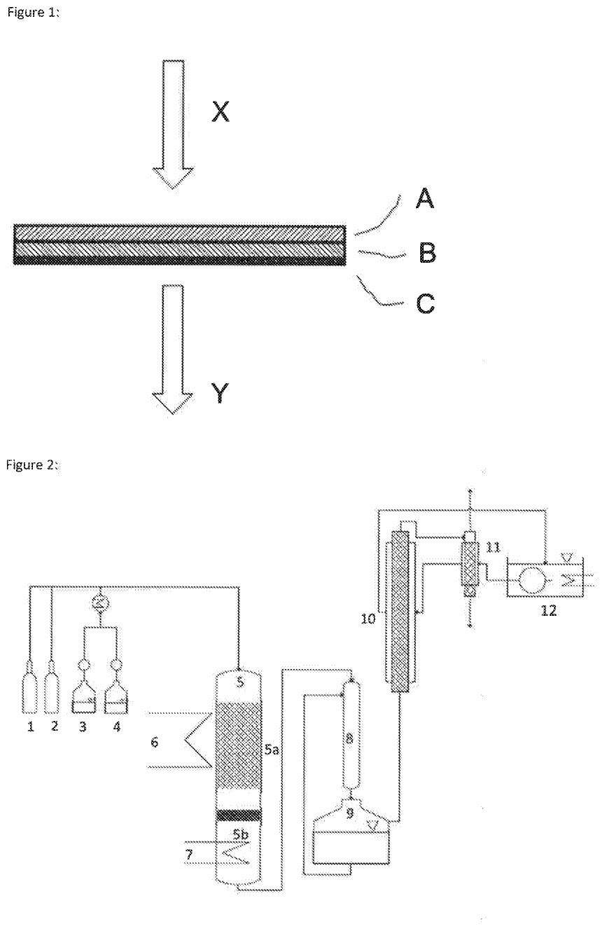

### EXAMPLES

Detailed Description of the Experimental Apparatus and Procedure

The experiments were carried out in an adiabatic mode of operation in a fused silica reactor having an internal diameter of 20 mm and filled with catalyst. The adiabatic operation of the reactor was achieved by passive insulation and dispenses completely with compensatory heating. The reactions were carried out using a gaseous water/methanol mixture (molar ratio of water/methanol: 1.0), air (410 standard I/h) and nitrogen (150 standard I/h) in such amounts that the molar ratio of methanol to oxygen was 2.5. This mixture was heated to 140° C. in a preheater located upstream of the reactor and passed through the reactor.

When metering rates and preheated temperature are set as described above, the catalyst bed usually attains, when the adiabatic reaction has been ignited, temperatures in the range from 590° C. to 710° C. The space velocity is typically in the range from 85 000 h⁻¹ to 120 000 h⁻¹. The product mixture exiting from the catalyst bed is cooled directly to 120° C. in a heat exchanger. The composition of the product mixture is determined by gas-chromatographic analysis.

The methanol conversion is defined as the molar amount of methanol reacted divided by the molar amount of methanol used. The formaldehyde selectivity is defined as the molar amount of formaldehyde formed divided by the molar amount of methanol reacted. The input number, as measure of the selectivity of the reaction of methanol to form formaldehyde at a given conversion, is defined as the amount of methanol in kilogram which has to be fed into the reactor system for 1.00 kilogram of formaldehyde to be produced in the reactor. The initial activation of the catalyst and the pressure drop which increases over the later course of time lead to the input number going through an optimum (minimum) over the time on stream. In the following examples and comparative examples, the average input number, the average methanol conversion and the average formaldehyde selectivity are determined cumulatively over a period of 18.5 days after attainment of the respective input number optimum.

For starting up the catalyst, a methanol/water/air/nitrogen mixture was heated to 300° C. in order to ensure ignition of the adiabatic reaction over the silver catalyst, with the molar ratio of methanol/oxygen being 7:1 and the introduction of nitrogen being 300 standard I/h at these temperatures. The adiabatic ignition occurred in the temperature range of 300° C.-350° C. The above-mentioned composition of water/methanol/air/nitrogen was subsequently metered in stepwise.

The temperature measurement was carried out by means of temperature sensors which were installed distributed over the cross section in the catalyst bed.

The pressure difference is recorded by means of pressure sensors upstream and downstream of the reactor. The temperature of the catalyst is controlled via the amount of air fed in.

### Comparative Example 1

### Two-Layer Catalyst with Granular Material Composed of Electrolytic Silver

For this experiment, the reactor was filled with a two-layer catalyst bed. The lower layer consists of a granular material composed of electrolytic silver having a particle size in the range from 1 to 2 mm and has an average thickness of 25 mm. The purity of the silver is 99.99% and the weight per unit area of this layer is 34 kg/m². The upper layer consists of a granular material composed of electrolytic silver having a particle size in the range from 0.5 to 1 mm and has an average thickness of 5 mm. The purity of this silver is 99.99% and the weight per unit area of this layer is 10.8 kg/m². The space velocity over the catalyst is 100 000 h⁻¹. The average inflow velocity is 1.12 m/s. The initial pressure drop over the reactor is 65 mbar. The rate of increase of the pressure drop is 0.81 mbar per day. The average input number is 1.214, the average methanol conversion is 96.6% and the average formaldehyde selectivity is 90.9%, in each case determined cumulatively for a period of 18.5 days, commencing at the point in time of the input number optimum, 0.5 days after commencement of formaldehyde production.

### Comparative Example 2

### Single-Layer Catalyst with Silver Wire Knitted

For this experiment, the reactor was filled with layers of silver wire knitted. The wire knitted consists of silver wires having a wire diameter of 50 μm. The purity of the silver is 99.99% and the weight per unit area of this layer is 18.4 kg/m². The layers of silver wire knitted have a total thickness of 5 mm. The space velocity over the catalyst is 800 000 h⁻¹. The average inflow velocity is 1.12 m/s. The initial pressure drop over the reactor is 88 mbar. The rate of increase of the pressure drop is 12.23 mbar per day. The average input number is 1.213, the average methanol conversion is 96.6% and the average formaldehyde selectivity is 91.0%, in each case determined cumulatively for a period of 18.5 days, commencing at the point in time of the input number optimum, 7.4 days after commencement of formaldehyde production.

### Example 1

### Two-Layer Catalyst with Silver Wire Knitted and Granular Material Composed of Electrolytic Silver

For this experiment, the reactor was filled with a two-layer catalyst bed. The lower layer consists of granular material composed of electrolytic silver having a particle size in the range from 1 to 2 mm and has an average thickness of 20 mm. The purity of the silver is 99.99% and the weight per unit area of this layer is 22.7 kg/m². The upper layer consists of individual layers of a silver wire knitted having a wire diameter of 50 μm and a total thickness of 0.5 mm. The purity of the silver is 99.99% and the weight per unit area of this layer is 1.8 kg/m². The space velocity over the catalyst is 220 000 h⁻¹. The average inflow velocity is 1.29 m/s. The initial pressure drop over the reactor is 60 mbar. The rate of increase of the pressure drop is 0.27 mbar per day. The average input number is 1.211, the average methanol conversion is 97.3% and the average formaldehyde selectivity is 90.5%, in each case determined cumulatively for a period of 18.5 days, commencing with the point in time of the input number optimum, 4.9 days after commencement of formaldehyde production.

### Example 2

### Two-Layer Catalyst with Silver Wire Gauze and Granular Material Composed of Electrolytic Silver

For this experiment, the reactor was filled with a two-layer catalyst bed. The lower layer consists of granular material composed of electrolytic silver having a particle size in the range from 1 to 2 mm and has an average thickness of 20 mm. The purity of the silver is 99.99% and the weight per unit area of this layer is 22.7 kg/m². The upper layer consists of two superposed woven gauzes made of silver wire having a wire diameter of 100 μm and has a total thickness of 2 mm. The mesh opening of the gauze is 25 mesh, the purity of the silver is 99.99% and the weight per unit area of this layer is 3.3 kg/m². The space velocity over the catalyst is 190 000 h⁻¹. The average inflow velocity is 1.12 m/s. The initial pressure drop over the reactor is 48 mbar. The rate of increase of the pressure drop is 0.55 mbar per day. The average input number is 1.213, the average methanol conversion is 97.2% and the average formaldehyde selectivity is 90.5%, in each case determined cumulatively for a period of 18.5 days, commencing at the point in time of the input number optimum, 27.5 days after commencement of formaldehyde production.

1.-12. (canceled)

13. A silver-comprising catalyst system comprising
a first catalyst layer A composed of a silver-comprising material which is present in a form consisting of wires and selected from the group consisting of balls of wire, gauzes and knitteds and
a second catalyst layer B composed of a silver-comprising material which is present in the form of granular material having an average particle size in the range from 0.5 to 5 mm,
with the catalyst layer B being in direct contact with the catalyst layer A,
wherein the silver-comprising material of the catalyst layer A has
a weight per unit area in the range from 0.3 to 10 kg/m² for the entire catalyst layer A and
a wire diameter in the range from 30 to 200 μm.

14. The silver-comprising catalyst system according to claim 13, wherein the silver-comprising material of the catalyst layer A has a weight per unit area in the range from 0.3 to 3 kg/m² for the entire catalyst layer A.

15. The silver-comprising catalyst system according to claim 13, wherein the silver-comprising material of the catalyst layer A has a wire diameter in the range from 30 μm to 150 μm.

16. The silver-comprising catalyst system according to claim 13, wherein the balls of wire, gauzes and knitteds of the catalyst layer A consist of wires which are silver-comprising fibers or threads having an essentially circular cross section.

17. The silver-comprising catalyst system according to claim 13, wherein the silver-comprising material of the catalyst layer B has an average particle size in the range from 0.75 mm to 4 mm.

18. The silver-comprising catalyst system according to claim 13, wherein the silver-comprising material of the catalyst layer A has a silver content of >98% by weight.

19. The silver-comprising catalyst system according to claim 13, wherein the silver-comprising material of the catalyst layer B has a silver content of >30% by weight.

20. An activated silver-comprising catalyst system obtainable by reacting the catalyst system according to claim 13 under operating conditions, wherein a feed stream comprising one or more C₁-C₁₀-alcohols and one or more oxidizing agents is passed at a temperature in the range from 350° C. to 750° C., a space velocity in the range from 36 000 h⁻¹ to 1 800 000 h⁻¹ and an inflow velocity in the range from 0.1 m s⁻¹ to 15 m s⁻¹ through the catalyst system or, under Activation conditions, a mixture comprising oxygen and an oxidizable material is passed at a temperature in the range from 350° C. to 750° C., a space velocity in the range from 36 000 h⁻¹ to 1 800 000 h⁻¹ and an inflow velocity in the range from 0.1 m s⁻¹ to 15 m s⁻¹ through the catalyst system, where the space velocity is the ratio of the volume stream which flows through the catalyst bed to the catalyst volume in the reactor.

21. The activated silver-comprising catalyst system according to claim 20 after at least two days of operation under operating conditions or activation conditions.

22. The activated silver-comprising catalyst system according to claim 20, wherein, during the reaction, at least 50% by weight of the silver-comprising material of the catalyst layer A ha.s been converted into a finely structured silver layer on the silver-comprising material of the catalyst layer B.

23. A process for the preparation of C₁-C₁₀-aldehydes and/or -ketones by oxidative dehydrogenation of the corresponding C₁-C₁₀-alcohols, wherein a feed stream comprising one or more C₁-C₁₀-alcohols and one or more oxidizing agents is passed at temperatures in the range from 350 to 750° C. through one or more catalyst systems according to claim 13 in such a way that the feed stream flows firstly through the catalyst layer A and immediately afterward through the catalyst layer B of the one or more catalyst systems.

24. The process according to claim 23, wherein formaldehyde is the C₁-C₁₀-aldehyde formed, methanol is the C₁-C₁₀-alcohol used and the feed stream is passed at temperatures in the range from 550 to 750° C. through the one or more catalyst systems according to the invention.
